(19) United States
(12) Patent Application Publication
(10) Pub. No.: US 2003/0129593 A1
(43) Pub. Date: Jul. 10, 2003

(54) Process for producing multiple oligonucleotides on a solid support

(75) Inventors: Richard T. Pon, Calgary (CA); Yogesh Sanghvi, Encinitas, CA (US); Shuyuan Yu, Calgary (CA)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(73) Assignee: University Technologies International Inc.

(21) Appl. No.: 09/946,172

(22) Filed: Sep. 5, 2001

Related U.S. Application Data
(60) Provisional application No. 60/229,532, filed on Sep. 5, 2000.

(51) Int. Cl. C12Q 1/68; C07H 21/04; C08G 63/48

(52) U.S. Cl. 435/6; 525/54.5; 536/25.3

(57) ABSTRACT

A process for production of the two or more molecules of interest. The present process may be advantageously used to produce two, three, four or more molecules of interest (the same or different from one another) on a single support material. A preferred embodiment comprises use of the present process to produce two or more oligonucleotides of interest. The process can be readily extended to other applications such combinatorial chemistry, peptide synthesis and the like. Various novel intermediates in the process are also disclosed.

### FIELD OF THE INVENTION

In one of its aspect, the present invention relates to a process for producing two or more molecules of interest on the same solid support material. In another of its aspects, the present invention relates to a process for producing two or more oligonucleotides on the same solid support material. Other aspects of the present invention relate to novel intermediates useful in the present process.

### DESCRIPTION OF THE ART

The art of conducting organic chemistry on solid supports is generally known. A useful review article on this topic may be found in “Organic Chemistry on Solid Supports” by Früchtel et al., Angew. Chem. Int. Ed. Engl., 1996, 35, pgs. 17-42.

As discussed in Früchtel et al., the art has developed automated solid-phase synthesis of polypeptides, oligonucleotides and oligosaccharaides. Of preferred and particular interest here is solid-phase synthesis of oligonucleotides and modified oligonucleotides. The following are useful review articles/textbooks on this topic:

Ilyer et al., Curr. Opin. Molec. Therap., 1999, 1, pgs. 344-358;

Verma et al., Annu. Rev. Biochem., 1998, 67, pgs. 99-134;

Montserra et al., Tetrahedron, 1994, 50, pg. 2617;

Beaucage et al., Tetrahedron, 1993, 49, pgs. 1925-1963;

Beaucage et al., Tetrahedron, 1993, 49, pgs. 6123-6194;

Beaucage et al., Tetrahedron, 1992, 48, pg. 2223;

Davis et al., Innovation and Perspectives in Solid Phase Synthesis (Ed.: R. Epton), Intercept, Andover, 1992, pg. 63;

Englisch et al., Angew. Chemie Intl. Ed. Engl., 1991, pgs. 613-629; and

Goodchild, Bioconjugate Chemistry, 1990, 1, pgs. 165-187.

In the solid-phase synthesis of oligonucleotides, it is known to synthesize the oligonucleotide on an inorganic solid support bearing a succinyl linker arm—see, for example, any of the following references:

Caruthers et al., Genetic Engineering, Plenum Press, New York (1982), Vol. 4, pgs. 1-17;

Letsinger et al., Genetic Engineering, Plenum Press, New York (1985), Vol. 5, pg. 191;

Froehler et al., Nucleic Acids Research, 14:5399-5407 (1986); and

Matteucci et al., Journal of American Chemical Society, 103:3185-3186 (1981).

Typically, the succinyl linker arm has the following general formula:

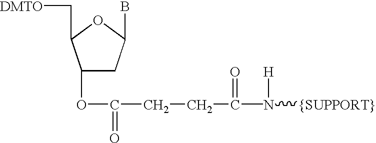

Thus, the succinyl group links the growing oligonucleotide from its terminal 3′-hydroxyl group by an ester bond to a primary amine on the support, which may be, for example, conventional controlled pore glass (CPG) or silica, by an amide bond. Once the desired oligonucleotide has been synthesized, it is freed or cleaved from the succinyl linker arm hydrolyzing the ester carbonyl group. The hydrolysis agent is usually concentrated ammonium hydroxide. Typically, this reaction can take from 1-4 hours to complete. With improvements to current solid-phase oligonucleotide synthesizers, this cleavage step can represent 50% or more of the total time require to synthesize the desired oligonucleotide.

Other linker arms for use in solid-phase oligonucleotide synthesis are also known. See, for example, U.S. Pat. No. 5,112,962 [Letsinger et al.] wherein there is taught an oxalyl linker arm.

In published International patent application WO 97/23497 [Pon et al. (Pon #1)], there is taught an improved linker arm for solid support oligonucleotide synthesis. The linker arm comprises the following formula:

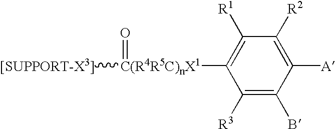

wherein: X¹ is selected from the group consisting of —O—, —S—, —S(O)₂—, —C(O)— and —N(R¹²)—; R¹² is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkaryl group, X³ is —O— or —N(H)—; R¹, R², R³, R⁴ and R⁵ are the same or different and are selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; n is 0, 1 or 2; and one of A′ and B′ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of A′ and B′ has the formula:

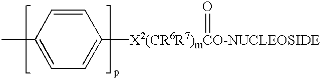

wherein X² is selected from the group consisting of —O—, —S—, —S(O)₂— and —N(R¹³)—; R¹³ is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkaryl group; R⁶ and R⁷ are the same or different and are selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; p is 0 or 1; and m is 0, 1 or 2. A process for producing the linker arm is also disclosed. The linker arm taught in Pon #1 is useful in solid support oligonucleotide synthesis and is characterized by a desirable combination of stability against spontaneous hydrolysis and ease of intentional cleavage of the synthesized oligonucleotide from the linker arm. A preferred linker arm in Pon #1 is hydroquinone-O,O′-diacetic acid. As is known in the art, this compound has the following structure:

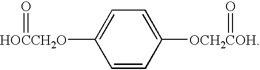

In published International patent application WO 97/23496 [Pon et al. (Pon #2)] there is taught a solid support for oligonucleotide synthesis. The solid support has the following formula:

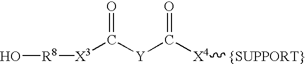

wherein: R⁸ is selected from the group consisting of a substituted or unsubstituted C₁-C₂₀ alkyl group, a substituted or unsubstituted C₅-C₃₀ aryl group and a substituted or unsubstituted C₅-C₄₀ alkylaryl group; X³ and X⁴ are the same or different and are selected from the group consisting of —O—, —S—, —S(O)₂— and —N(R¹²)—; R¹² is selected from the group consisting of a substituted or unsubstituted C₁-C₂₀ alkyl group, a substituted or unsubstituted C₅-C₃₀ aryl group and a substituted or unsubstituted C₅-C₄₀ alkylaryl group; and Y is selected from the group consisting of:

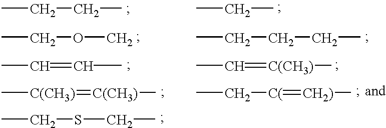

The derivatized solid support is characterized by being reusable in an otherwise conventional oligonucleotide production protocol.

In published International patent application WO 00/01711 [Pon et al. (Pon #3)] there is taught a solid support for oligonucleotide synthesis. The solid support has the following formula:

Z-O-T [symbol] [SUPPORT]

wherein Z is a linker moiety and T is an organic radical. The solid support taught by Pon #3 is a reusable support which is an improvement over that in Pon #2.

While the teachings of Pon #1, Pon#2 and Pon #3 represent significant advances in the art, there is still room for improvement.

The synthesis of oligonucleotide sequences by the sequential coupling of nucleoside phosphoramidites (or other activated nucleotide derivatives) on solid-phase supports has become well established. Generally, however, this method is limited to the production of either a single oligonucleotide sequence or a degenerate mixture sharing a single common consensus sequence (which is used as a single product) per synthesis. To date, demand for increased oligonucleotide production has been met by methods which produce multiple different oligonucleotides in parallel synthetic runs (e.g., by parallel synthesis in 96 well trays) or which use process scale instrumentation to produce individual oligonucleotides in larger (millimole) quantities. These methods still produce only one product (e.g., one oligonucleotide) per synthesis.

An alternative pproach to increasing the number and/or the amount of oligonucleotide produced is to produce multiple oligonucleotides linked in a serial or tandem fashion. This approach has been described by McLean et. al. (1994, Nucleic Acids Res. 22, 2998-3004; U.S. Pat. No. 5,552,535 and U.S. Pat. No. 5,393,877), who teach phosphoramidite-based linking reagents. In this strategy, a “universal” non-nucleotide based linker molecule is used to convert the terminal end (usually the 5′-end) of one immobilized oligonucleotide into a starting point for a new synthesis. This approach purportedly is suitable for preparing combinations of different sequences since only a single universal linking reagent is required. However, cleavage of the linked products requires longer and harsher deprotection conditions to produce sequences with 3′-nonphosphorylated ends (these are needed for most applications).

Thus, despite the advances made in the art, there is an ongoing need to have a relatively simple mode by which multiple oligonucleotides (or other molecules of interest) and the like can be produced with a variety of linking componds and/or with a variety of solid-phase support materials. It would be particularly advantageous if the so-produced multiple oligonucleotides (or other molecules of interest) could be cleaved in a controlled facile manner.

### SUMMARY OF THE INVENTION

It is an object of the present invention to provide a novel process for producing multiple molecules of interest (e.g., oligonucleotides) on a solid support material.

It is another object of the present invention to provide a novel intermediate useful for producing multiple molecules of interest (e.g., oligonucleotides) on a solid support material.

Accordingly, in one of its aspects, the present invention provides a process for producing at least two molecules of interest on a solid support material, the process comprising the steps of:

(i) reacting a support having Formula I:

[SUPPORT]-Z¹-Y¹ (I)

wherein:

Z¹ is N or O;

and Y¹ is —H when Z¹ is O or Y¹ is —(R)(H) when Z¹ is N wherein R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group;

with a compound having Formula II:

H-A¹-B¹ (II)

wherein:

A¹ is a linker moiety having the formula

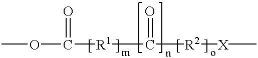

wherein:

R¹ and R² are the same or different and each is an organic moiety (e.g., a C₁-C₃₀₀ organic moiety);

m is 0 or 1;

n is 0 or 1;

o is 0 or 1; and

X is O or N(R) wherein R is as defined above; and

B¹ is hydrogen or a protecting group;

to produce a material having Formula III:

[SUPPORT]-Z¹-A¹-B¹ (III)

(ii) conducting at least one synthetic reaction on the material having Formula III to produce a material having Formula IV:

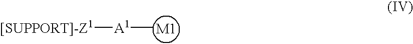

wherein M1 is a precursor to one of the at least two molecules of interest;

(iii) reacting the material of Formula IV with a compound having Formula V:

H-A²-B² (V)

wherein:

A² is a moiety selected from the group defined above for A¹; and

B² is a moiety selected from the group defined above for B¹;

to produce a material having Formula VI:

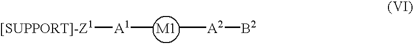

(iv) conducting at least one synthetic reaction on the material having Formula VI to produce a material having Formula VII:

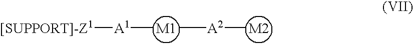

wherein M2 is a precursor of the other of the at least two molecules of interest; and

(v) subjecting the material of Formula VII to a cleavage step to release at least one of the at least two molecules of interest from the support material.

In another of its aspects, the present invention provides a process for producing at least two oligonucleotides on a solid support material, each oligonucleotide having a predetermined sequence, the process comprising the steps of:

(i) reacting a support having Formula I:

[SUPPORT]-Z¹-Y¹ (I)

wherein:

Z¹ is N or O;

and Y¹ is —H when Z¹ is O or Y¹ is —(R)(H) when Z¹ is N wherein R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group;

with a compound having Formula II:

H-A¹-B¹ (II)

wherein:

A¹ is a moiety having the formula

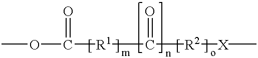

wherein:

R¹ is an organic moiety;

R² is a nucleoside moiety or a nucleotide moiety;

m is 0 or 1;

n is 0 or 1;

o is 0 or 1; and

X is O or N(R) wherein R is as defined above; and

B¹ is hydrogen or a protecting group;

to produce a material having Formula III:

[SUPPORT]-Z¹-A¹-B¹ (III)

(ii) conducting at least one synthetic reaction on the material having Formula III to produce a material having Formula IV:

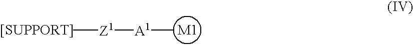

wherein M1 comprises at least a portion of the predetermined sequence of one of the at least two oligonucleotides of interest;

(iii) reacting the material of Formula IV with a compound having Formula V:

H-A²-B² (V)

wherein:

A² is a moiety selected from the group defined above for A¹; and

B² is a moiety selected from the group defined above for B¹;

to produce a material having Formula VI:

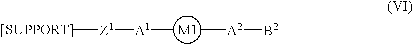

(iv) conducting at least one synthetic reaction on the material having Formula VI to produce a material having Formula VII:

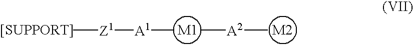

wherein M2 comprises at least a portion of the predetermined sequence of the other of the at least two oligonucleotides of interest; and

(v) subjecting the material of Formula VII to a cleavage step to release at least one of the at least two oligonucleotides of interest from the support material.

In another of its aspects the present invention provides a material having Formula VI:

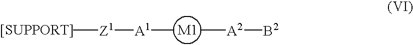

wherein:

Z¹ is O or N(R) wherein R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group;

A¹ and A² are the same or different and each is a linker moiety having the formula

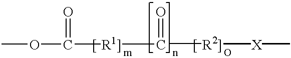

wherein:

R¹ and R² are the same or different and each is an organic moiety;

m is 0 or 1;

n is 0 or 1;

o is 0 or 1; and

X is O or NR wherein R is as defined above;

B² is hydrogen or a protecting group; and

M1 is a precursor to a first molecule of interest.

In yet another of its aspects, the present invention provides a material having Formula VII:

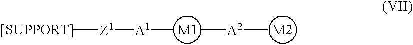

wherein:

Z¹ is O or N(R) wherein R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group;

A¹ and A² are the same or different and each is a linker moiety having the formula

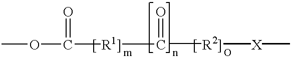

wherein:

R¹ and R² are the same or different and each is an organic moiety;

m is 0 or 1;

n is 0 or 1;

o is 0 or 1; and

X is O or NR wherein R is as defined above;

B² is hydrogen or a protecting group; and

M1 is a precursor to a first molecule of interest; and

M2 is a precursor to a second molecule of interest.

In yet another of its aspects, the presentin invention provides process for producing at least two oligonucleotides on a solid support material, each oligonucleotide having a predetermined sequence, the process comprising the steps of:

(i) conducting at least one synthetic reaction on a material having Formula III

[SUPPORT]-Z¹-A¹-B¹ (III)

wherein:

Z¹ is O or N(R) wherein R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group;

A¹ is a moiety having the formula

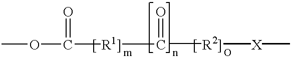

R¹ is an organic moiety;

R² is a nucleoside moiety or a nucleotide moiety;

m is 0 or 1;

n is 0 or 1;

o is 0 or 1; and

X is O or N(R) wherein R is as defined above; and

B¹ is hydrogen or a protecting group;

to produce a material having Formula IV:

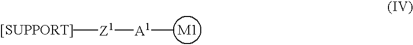

wherein M1 comprises at least a portion of the predetermined sequence of one of the at least two oligonucleotides of interest;

(ii) reacting the material of Formula IV with a compound having Formula V:

H-A²-B² (V)

wherein:

A² is a moiety selected from the group defined above for A¹; and

B² is a moiety selected from the group defined above for B¹;

to produce a material having Formula VI:

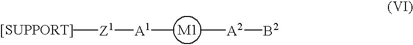

(iii) conducting at least one synthetic reaction on the material having Formula VI to produce a material having Formula VII:

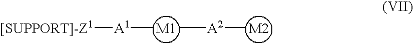

wherein M2 comprises at least a portion of the predetermined sequence of the other of the at least two oligonucleotides of interest; and

(iv) subjecting the material of Formula VII to a cleavage step to release at least one of the at least two oligonucleotides of interest from the support material.

As used throughout this specification, the term “oligonucleotide” is intended to have a broad meaning and encompasses conventional oligonucleotides, backbone-modified oligonucleotides (e.g., phosphorothioate, phosphorodithioate and methylphophonate analogs useful as oligotherapeutic agents), labeled oligonucleotides, sugar-modified oligonucleotides and oligonucleotide derivatives such as oligonucleotide-peptide conjugates.

### BRIEF DESCRIPTION OF THE DRAWING

Embodiments of the present invention will be described with reference to the accompanying drawing, in which:

FIG. 1 illustrates an exemplary illustration of a tandem olignucleotide synthesis using the present process.

### DETAILED DESCRIPTION OF THE PREFERRED EMBODIMENTS

An aspect of the present invention relates to a process for production of the two or more molecules of interest. Thus, the present process may be advantageously used to produce two, three, four or more molecules of interest (the same or different from one another) on a single support material.

A preferred embodiment comprises use of the present process to produce two or more oligonucleotides of interest. While much of this specification will refer to production of two or more oligonucleotides of interest, those of skill in the art will appreciate that the scope of the present invention can be readily extended to other applications such combinatorial chemistry, peptide synthesis and the like. Further details on these other molecules of interest may be found in one or more of the following references:

Lloyd-Williams et al., Chemical Approaches to the Synthesis of Peptides and Proteins, CRC Press, New York, 1997;

Hecht. Bioorganic Chemistry: Peptides and Proteins, Oxford University Press, New York, 1998;

Bunin, The Combinatorial Index, Academic Press, San Diego, 1998;

Booth et al., Tetrahedron, 1998, 54, pgs. 15385-15443;

Hijfte et al., J. Chromatography, 1999, B, 725, pgs. 3-15; and

Sofie, Molecular Diversity, 1998, 3, pgs. 75-94.

When the present invention is applied to the production of two or more oligonucleotides from a single support material, it is preferred to utilize the linker compounds, reagents, general reaction conditions and equipment taught in Pon #1, Pon #2 and Pon #3 referred to hereinabove.

Throughout this specification, when reference is made to a substituted moiety, the nature of the substitution is not specification restricted and may be one or more members selected from the group consisting of hydrogen, a C₁-C₂₀ alkyl group, a C₅-C₃₀ aryl group, a C₅-C₄₀ alkaryl group (each of the foregoing hydrocarbon groups may themselves be substituted with one or more of a halogen, oxygen and sulfur), a halogen, oxygen and sulfur.

Thus, in a preferred embodiment, Step (i) of the present process comprises use of a support having Formula I:

[SUPPORT]-Z¹-Y¹ (I)

wherein:

Z¹ is N or O;

and Y¹ is —H when Z¹ is O or Y¹ is —(R)(H) when Z¹ is N wherein R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group.

In above formula, the SUPPORT is a conventional solid support. The nature of the solid support is not particularly restricted and is within the purview of a person skilled in the art. Thus, the solid support may be an inorganic substance. Non-limiting examples of suitable inorganic substances may be selected from the group consisting of silica, porous glass, aluminosilicates, borosilicates, metal oxides (e.g., aluminum oxide, iron oxide, nickel oxide) and clay containing one or more of these. Alternatively, the solid support may be an organic substance such as a cross-linked polymer. Non-limiting examples of a suitable cross-linked polymer may be selected from the group consisting of polyamide, polyether, polystyrene and mixtures thereof. One preferred solid support for use herein is conventional and may be selected from controlled pore glass beads and polystyrene beads. Another preferred support is the reusable support taught in Pon #2 and Pon #3 referred to hereinabove.

In Step (i) of the preferred embodiment, of the present process, the support is reacted with a compound having Formula II:

H-A¹-B¹ (II)

wherein:

A¹ is a moiety having the formula

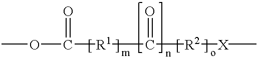

wherein:

R¹ is an organic moiety;

R² is a nucleoside moiety or a nucleotide moiety;

m is 0 or 1;

n is 0 or 1;

o is 0 or 1; and

X is O or NR wherein R is as defined above; and

B¹ is hydrogen or a protecting group.

The compound of Formula II is a linker compound. While various conventional linker compounds may be used, it is preferred to use a linker compound having the formula:

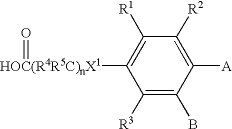

or derivate thereof, wherein: X¹ is selected from the group consisting of —O—, —S—, —S(O)₂—, —C(O)— and —N(R¹²)—; R¹² is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkaryl group; R¹, R², R³, R⁴ and R⁵ are the same or different and are selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; n is 0, 1 or 2; and one of A and B is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of A and B has the formula:

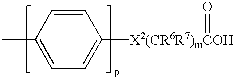

wherein p is 0 or 1, X² is selected from the group consisting of —O—, —S—, —S(O)₂—, —C(O)— and —N(R¹³)—; R¹³ is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkaryl group, R⁶ and R⁷ are the same or different and are selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkaryl group, and m is 0, 1 or 2.

Preferably, B in the above formula is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, thereby rendering the acid-containing moieties in a “para” relationship.

Preferably, both R⁴ and R⁵ in above preferred formula are hydrogen, and both R⁶ and R⁷ in the above preferred formula are hydrogen. More preferably, each of R⁴, R⁵, R⁶, R⁷, R¹² and R¹³ in the above preferred formula are hydrogen.

Preferably, at least one, more preferably both, m and n in the above preferred formula are 1, and p in the above formula is 0.

Preferably, each of R¹, R² and R³ in the above preferred formula is hydrogen, and X¹ and X² in the above preferred formula are both O.

The preferred linking compound for use in Step (I) of the present process is hydroquinone-O,O′-diacetic acid. As is known in the art, the compound has the following structure:

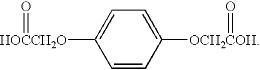

Alternatively, one could start with a nucleoside having this preferred linking compound already attached to the 3′-OH position, i.e., a protected nucleoside-3′-O-hydroquinone-O,O′-diacetic acid hemiester.

Next, the oligonucleotide of desired sequence may be produced using conventional techniques. See, also Pon #1, Pon #2, Pon #3 and the review articles/textbooks referred to hereinabove.

A preferred method for production of deoxyribonucleotides in the context of a preferred embodiment of the present process is to use a nucleoside with a 5′-dimethoxytrityl protecting group and an appropriate exocyclic amino protecting group, e.g., N⁶-benzoyl-5′-dimethoxytrityl-2′-deoxyadenosine, N⁴-benzoyl-5′-dimethoxytrityl-2′-deoxycytidine, 5′-dimethoxytrityl-N²-isobutyryl-2′-deoxyguanosine, or 5′-dimethoxytritylthymidine.

A preferred method for production of ribonucleotides in the context of a preferred embodiment of the present invention is to use a 5′-dimethoxytrityl protected nucleoside, with appropriate exocyclic amino protection, and no protecting groups on either of the 2′- or 3′-hydroxyl positions. The linker can then react with either one of the two adjacent hydroxyl groups (it does not matter which) to give a mixture of 2′- and 3′-linkages. The unreacted hydroxyl groups may then be acetylated by treatment of the immobilized nucleoside with acetic anhydride. Alternatively, ribonucleosides which have a 5′-dimethoxytrityl group, appropriate exocyclic amino group protection, and either a 3′-hydroxyl protecting group or a mixture of 2′- and 3′-protecting groups can be used. The 3′-protected compounds are generally unwanted isomers which are simultaneously produced when the 2′-hydroxyl position is protected and have little other use.

The use of protecting groups is conventional in the art and the selection thereof is within the purview of a person skilled in the art. Thus, it possible to utilize other protecting groups not specifically referred to in this specification without deviating from the scope of the present invention.

Thus, the first of the two oligonucleotides of interest is built up on the support material in a conventional manner. At this point, the synthesized oligonucleotide is not cleaved, unlike prior art approaches. Instead, the material is reacted with a linker compound of Formula V:

H-A²-B² (V)

wherein:

A² is a moiety selected from the group defined above for A¹; and

B² is a moiety selected from the group defined above for B¹.

As stated above a preferred linker compound for use in the present process is that taught in Pon #1. Of course, alternate linker compounds are also useful. Thus, R¹ in A¹ and A² generally is organic moiety. Preferably, the hydrocarbon moiety is a C₁₋₃₀₀ hydrocarbon moiety, optionally substituted with one or more of oxygen, nitrogen, halogen and sulfur. In some embodiments of the present invention, R² in A¹ and A² also is generally an organic moiety. Preferably the hydrocarbon moiety is a C₁₋₃₀₀ organic moiety, optionally substituted with one or more of oxygen, nitrogen, halogen and sulfur. Further, the term “alkyl”, as used throughout this specification, is intended to encompass hydrocarbon moieties having single bonds, doubles bonds, triple bond and mixtures thereof.

Thus, in one preferred embodiment, R¹ is a moiety having the formula:

—(R⁴R⁵C)ₙ—(Y)ₒ—(CR⁶R⁷)ₘ—

wherein R⁴, R⁵, R⁶ and R⁷ are the same or different and are selected from the group consisting of hydrogen, halogen, a substituted or unsubstituted C₁-C₂₀ alkyl group, a substituted or unsubstituted C₅-C₃₀ aryl group and a substituted or unsubstituted C₅-C₄₀ alkylaryl group, Y is selected from the group consisting of O, S, SO₂ and O—((CH₂)ₗ—O)_{q}, l is an integer less than or equal to 60, q is an integer in the range of 1-1000, n and m are the same or different and are 0, 1 or 2, and o is 0 or 1.

In another preferred embodiment, R¹ is a moiety selected from the group consisting of the following formulae:

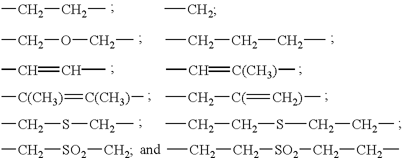

The second oligonucleotide of interest may then be built up on the same material in a manner similar to the build of the first oligonucleotide of interest. Further oligonucleotides of interest can be built up on the same support material. Build up of the oligonucleotides of interest on the same support material is subject to the general limitations of accessibility (e.g., support pore size), resistance to depurination, coupling efficiency and the like. Persons skilled in the art will readily recognize that these limitations are not severe and oligonucleotides, inter alia, consisting of 100 or more bases in total can be assembled with desirable yields.

At this point, once the oligonucleotides of interest have been synthesized one, some or all of them may be cleaved with respect to the support material. The cleavage step usually comprises hydrolysis at the point of attachment of the initial nucleoside to the linking compound.

The reagent used to effect cleavage is not particularly restricted and is within the purview of a person skilled in the art. Preferably, the reagent is a base. Non-limiting examples of suitable reagents for this purpose may be selected from the group consisting of ammonia, ammonium hydroxide, ammonium hydroxide/methanol, triethylamine/alcohol (e.g., ethanol, methanol, etc.), methylamine, dimethylamine, trimethylamine/water, methylamine/ammonium hydroxide, ammonia/methanol, potassium carbonate/methanol, t-butylamine, ethylenediamine and the like.

Cleavage can also be achieved using a solution of 20% piperidine in DMF at room temperature. Significantly, however, the rate of cleavage is slow (t_{1/2}˜200 min) and so piperidine solutions can still be used to remove more sensitive protecting groups (such as the fluorenylmethoxycarbonyl (Fmoc) group) or to convert underivatized carboxylic acid groups into unreactive amides. The linker arm may also be cleaved under neutral conditions by treatment with room temperature fluoride ion (e.g., 1M tetrabutylammonium fluoride/THF or triethylamine trihydrofluoride).

The preferred cleavage method is treatment with concentrated aqueous ammonium hydroxide for either 3 or 15 minutes, respectively, for phosphodiester or phosphorothioate oligonucleotides.

In the present process, the linker compounds of Formula II and Formula V may be the same or different. This will depend on the nature of the oligonucleotides being produced.

Thus, if the first oligonucleotide of interest and the second oligonucleotide of interest have identical sequences, it is preferred to use the same linker compound in Formulae II and V. Once the multiple sequences have been produced, a single cleavage step may be used to separate all oligonucleotides concurrently from the support material.

Alternatively, if the first oligonucleotide of interest and the second oligonucleotide of interest have different sequences, it is preferred to use different linker compounds in Formulae II and V. Once the multiple sequences have been produced, sequential cleavage steps may be used to selectively separate one oligonucleotide from the support material and thereafter separate the other oligonucleotide from the support material. This facilitates separation of the two oligonucleotides from each other. Of course, if such separation of the two oligonucleotides is not required for their intended application, it is advantageous to use reagents which facilitate concurrent cleavage of all oligonucleotides from the support material.

Thus, a distinct advantage of the present invention is the ability to reduce the number of instrument runs (by one half or more) required to produce a large number of oligonucleotides (or other molecules of interest).

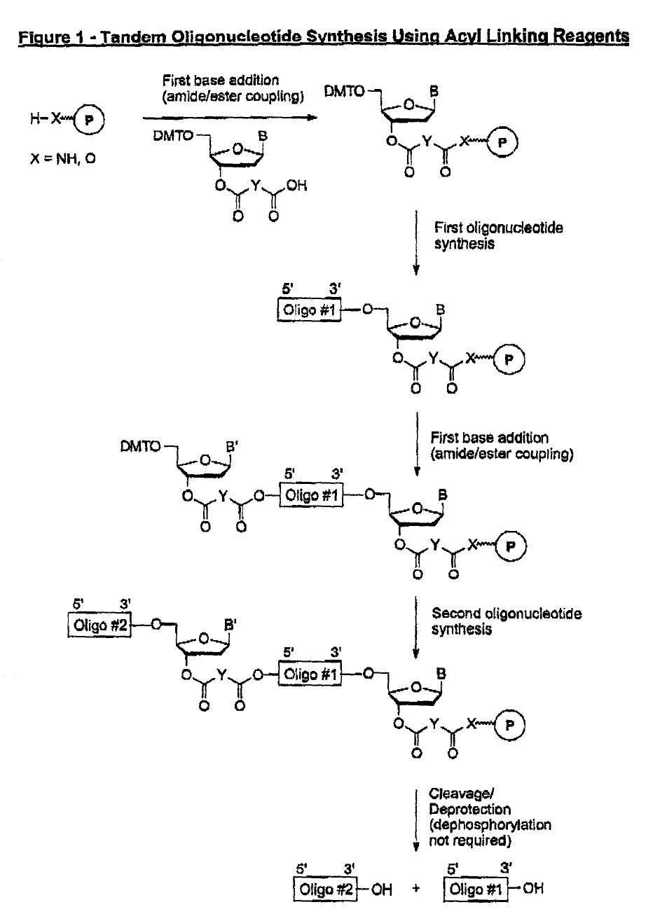

FIG. 1 illustrates an exemplery illustration of a tandem olignucleotide synthesis using the present process.

Embodiments of the invention will be illustrated in the following Examples which should not be construed as limiting the scope of the invention.

Equipment and Reagents

DNA Synthesizer

A four column Perkin-Elmer Applied Biosystems model 394 DNA synthesizer with eight base ports was used. A custom “Begin” procedure (see printout of procedure Load1234&Cap set out in Appendix A) was created to deliver the nucleoside hemiester and nucleoside coupling reagents to the synthesis column (10 minute coupling time to derivatize the support or oligonucleotide terminus with a nucleoside) and to cap (5 minutes) off any unreacted sites.

Capillary Electrophoresis

A Hewlett-Packard 3D CE instrument was used to perform analysis with a HP oligonucleotide capillary gel electrophoresis analysis kit.

Nucleoside Hemiester Reagent

5′-dimethoxytrityl-N⁶-benzoyl-2′-deoxyadenosine-3′-O-hydroquinone diacetyl hemiester or 5′-dimethoxytrityl-N⁴-benzoyl-2′-deoxycytidine-3′-O-hydroquinone diacetyl hemiester (0.45 mmol, 425 mg) and diisopropylethylamine (0.45 mmol, 78 μL) were dissolved in anhydrous acetonitrile (3 mL), filtered, and installed on bottle position #7 of the PE/ABD 394 DNA synthesizer.

Nucleoside Coupling Reagent

O-Benzotriazol-1-yl-N,N,N′,N′-tetramethyluronium hexafluorophosphate (HBTU, 0.60 mmol, 227 mg) and 4-dimethylaminopyridine (DMAP, 0.60 mmol, 73 mg) were dissolved in anhydrous acetonitrile (4 mL), filtered, and installed on bottle position #8 of the PE/ABD 394 DNA synthesizer.

Chloroacetic Anhydride Capping Reagents

1 M chloroacetic anhydride in THF (Cap A) and 1M lutidine/2M N-methylimidazole/THF (Cap B) were substituted for the conventional Cap A (1M acetic anhydride/1M lutidine/THF) and Cap B (2M N-methylimidazole/THF) capping reagents as specified in Example 3 hereinbelow.

Sulfurization Reagent.

0.05 M Beaucage reagent in acetonitrile was used instead of the regular iodine/water oxidation reagent in Example #3 hereinbelow

In the following Examples the amount of nucleoside (loading) on the insoluble supports was determined by spectrophotometric trityl analysis. In this procedure, a sample of support (4-5 mg) was accurately weighed directly into a 10 mL volumetric flask. A solution of dichloroacetic acid in 1,2-dichloroethane in a volume ratio of 5:95 was then added to fill the flask. The contents were then thoroughly mixed and the absorbance of the orange coloured solution was measured at 503 nm using a Philips UV/Vis spectrophotometer. The nucleoside loading (in μmol/g of CPG) was then calculated as:

Loading=(A₅₀₃×Vol×1000)/(Wt×76)

wherein A₅₀₃=absorbance at 503 nm, Vol=solution volume in ml, and Wt=amount of CPG tested in mg. The accuracy of the trityl determination was approximately ±2-3%.

### EXAMPLE 1

### Synthesis of Multiple Hexanucleotides on LCAA-CPG

In this Example, the increased amount of phosphodiester hexanucleotide dAAAAA, which can be obtained by three tandem syntheses relative to a single synthesis is demonstrated.

Two synthesis columns (˜0.2 μmole scale) were prepared using 500A long chain alkylamine controlled pore glass (LCAA-CPG) support which had previously been derivatized with 30 μmol/g of 5′-dimethoxytrityl-N⁶-benzoyl-2′-deoxyadenosine-3′-O-hydroquinone diacetic acid. The columns were used with a standard 0.2 μmole scale synthesis cycle and regular synthesis reagents. The exact weight of support and the amount of starting nucleoside in each column was recorded.

The first column was used as a control and a single dA₆ oligonucleotide was prepared. This was cleaved from the support (NH₄OH, room temperature, 5 minutes) and deprotected (NH₄OH, 16 hours, 55° C.). This yielded 16.6 A₂₆₀ units of crude product which was 1,580 A₂₆₀ units per gram of support or 63 A₂₆₀ units per μmole of initial nucleoside.

The second column had a dA₆ sequence synthesized thereon in the same manner. However, after synthesis the product was left on the support (DMT off). The Load1234&Cap custom begin procedure was then used to attach another dA nucleoside to the 5′-end of the first oligonucleotide. A second dA₆ synthesis was then performed (DMT off). The product was still left attached to the support and the Load1234&Cap custom begin procedure was run again and a third dA₆ sequence was synthesized (DMT off) to yield a support with three consecutive dA₆ sequences thereon. The products were then cleaved from the support (NH₄OH, room temperature, 5 minutes) and deprotected (NH₄OH, 16 hours, 55° C.). This yielded 61.7 A₂₆₀ units of crude product which was 5,460 A₂₆₀ units per gram of support or 226 A₂₆₀ units per μmole of initial nucleoside.

In comparison to the above control experiment, the amount of product obtained from a single synthesis column (i.e., the second column described above) was increased ˜3.5 fold.

Analysis of the unpurified products obtained from both syntheses was performed by capillary electrophoresis (CE) and both syntheses produced the same major product. The percent full length product in the single synthesis was 85.3% and the percent full length product in the triple tandem synthesis was 91.7%.

### EXAMPLE 2

### Synthesis of Multiple Oligonucleotides of Different Lengths on LCAA-CPG

In this Example, oligonucleotides of different length were prepared by tandem synthesis on the same insoluble support.

A synthesis column was used in a similar manner as described in Example 1 to consecutively prepare a dA₆ hexanucleotide, dA₁₀ decanucleotide, and a dA₁₄ tetradecanucleotide sequences on the same support. After completion of the last synthesis, the products were then cleaved from the support (NH₄OH, room temperature, 5 minutes) and deprotected (NH₄OH, 16 hours, 55° C.). This yielded 83.7 A₂₆₀ units of crude product which was 8,370 A₂₆₀ units per gram of support or 339 A₂₆₀ units per μmole of initial nucleoside.

Analysis of the unpurified product by CE showed the presence of the three expected sequences (dA₆, dA₁₀, and dA₁₄) as the major products.

### EXAMPLE 3

### Synthesis of Multiple Phosphorothioate Oligonucleotides on a Reusable Support (GLY-CPG)

In this Example, phosphorothioate modified anti-sense oligonucleotides of mixed base composition were prepared in tandem to increase the amount of material produced (i.e., relative to a single synthesis). Also, this Example illustrates that a reusable, hydroxyl derivatized support can be used and recycled to further increase the amount of product obtained from a given amount of support (see Pon #2 and Pon #3 referred to hereinabove for more information on reusable, hydroxyl derivatized supports).

Two synthesis columns (˜1 μmole scale) were prepared using 500 Å Glycerol derivatized controlled pore glass beads (GLY-CPG) which did not have any nucleoside attached to the surface. The control column (for single use) contained 13.1 mg and the experimental column (for multiple syntheses) contained 12.4 mg of support. The following procedure was then performed:

A. The regular capping reagents were replaced with the chloroacetic anhydride reagents.

B. The Load1234&Cap begin procedure was run to attach the first nucleoside (dA) to the support and to cap off any unreacted hydroxyl groups.

C. The chloroacetic anhydride capping reagents were removed and the regular (acetic anhydride) reagents were reinstalled.

D. The 20-mer sequence, dGCCCAAGCTGGCATCCGTCA, was synthesized, using a 1 μmole scale phosphorothioate synthesis cycle and the DMT-off/manual ending method. The initial amount of nucleoside attached to the support was determined by quantitative trityl analysis.

E. The Load1234&Cap begin procedure was then repeated (using regular acetic anhydride capping) to add another dA nucleoside to the end of the first oligonucleotide.

F. The 20-mer sequence, dGCCCAAGCTGGCATCCGTCA, was synthesized again, using the DMT-off/auto ending method. Cleavage from the support was performed using NH₄OH (15 minutes, room temperature). The released product was deprotected by heating (16 hours, 55°), evaporated to dryness, redissolved in water, and quantitated by UV at 260 nm.

G. The synthesis column was removed from the synthesizer, treated with 0.05 M K₂CO₃/methanol (5 minutes), rinsed with methanol (5 mL), dried by aspirator (5 minutes), and re-installed on the synthesizer.

This procedure was then repeated a total of five times on the column containing 12.4 mg of support. A single control synthesis, consisting only of steps A-D. Thereafter, cleavage and deprotection of the product was also performed. The results, provided in Table 1, illustrate that, each time the support was used in a tandem synthesis, the amount of product obtained, per micromole of starting nucleoside, was increased ˜35-90% relative to the single synthesis control experiment. In addition, because the support was re-used, a total of 955 A₂₆₀ units of material were obtained from only 12.4 mg of support (77,000 A₂₆₀ units/g). Since the control column (used once) produced only 143 A₂₆₀ units (10,900 A₂₆₀ units/g), the relative cost of the support was reduced by a factor of seven. Capillary electrophoresis was also used to compare the composition of the crude products from both the single synthesis control and the repetitive tandem syntheses.

### EXAMPLE 4

### Synthesis of Multiple 24 Base Long M13 Universal Sequencing Primers on 1,000 Å CPG

In this Example, a series of up to four multiple 24-mers, was prepared on medium loading, 1,000 Å CPG to demonstrate that tandem oligonucleotides of up to 96 bases in total length can be efficiently prepared on wide pore supports.

Four synthesis columns were filled with 2′-dDMT-C(Bz) derivatized medium loading (˜20 μmol/g) 1,000 Å CPG (ChemGenes, Waltham, Mass.). The first column was used as the single synthesis control (1×24-mer), the other columns were used to prepare double (2×24-mer), triple (3×24-mer), and quadruple (4×24-mer) tandem syntheses of the M13 universal sequencing primer, dCGCCAGGGTTTTCCCAGTCACGAC, on a 0.2 μmole scale. The general procedure described in Example 1 was followed with the following exceptions. First, the usual detritylation reagent (5% dichloroacetic acid, v/v) was replaced with a 2% dichloroacetic acid (v/v) solution to reduce depurination. Secondly, the NH₄OH cleavage step was increased from 5 minutes to 60 minutes because of the succinyl linker on the 1,000 Å CPG.

After each synthesis, the crude products were collected and deprotected. The amount of crude material was measured by UV at 260 m—see Table 2—and samples of each synthesis were analyzed by capillary electrophoresis. The results illustrate that the amount of product obtained, relative to a single synthesis, was increased by a factor of between 1.5 to 2.5 times, depending upon the number of consecutive tandem syntheses performed. Also, importantly, the CE analyses indicated that there was no significant change in the composition of the crude product, even when four consecutive 24-mers were prepared. Integration of the peak areas also showed that the amount of full length product in each of the three tandem syntheses was greater than in the single synthesis.

### EXAMPLE 5

### Nucleoside Coupling Using 2,4,6-Triisopropylbenzenesulphonyl Chloride (TPS-Cl) as the Coupling Reagent

This Example illustrates how formation of the ester linkage to the 5′-position of an immobilized nucleoside can also be performed using an arylsulphonyl chloride coupling reagent (TPS-Cl) and N-methylimidazole (NMI) instead of HBTU and DMAP. In this Example, the oligonucleotide synthesis steps were omitted and a dinucleoside, joined via a 3′-5′ hydroquinone diacetyl diester linkage, was prepared.

Two experiments containing recrystallized TPS-Cl (Aldrich, 15 mg) and N⁶-benzoyl-2′-deoxyadenosine derivatized LCAA-CPG (100 mg @ 53 μmol/g) were set up in small glass septum sealed vials and NMI (10 μL), anhydrous acetonitrile (100 μL) and 0.15 M 5′-dimethoxytrityl-N⁴-benzoyl-2′-deoxycytidine-3′-O-hydroquinone diacetyl hemiester/diisopropylethylamine solution (250 μL) were added to each vial. The mixtures were then shaken at room temperature (10 or 60 minutes), filtered off, washed sequentially with dichloromethane, methanol, and dichloromethane and dried. The amount of nucleoside coupled to the supports was then determined by trityl analysis to be 48 or 52 μmol/g, respectively for the 10 and 60 minutes coupling times. Thus, the yields from the TPS-Cl/NMI coupling reactions were 90% and 98%, respectively, for the 10 and 60 minute coupling times.

NB: The TPS-Cl/NMI coupling reagent has previously been used to form phosphotriester linkages in oligonucleotide synthesis (V. A. Efimov, S. V. Reverdattoo and O. G. Chakhmakcheva, 1982, Nucleic Acids Res. 10, 6675-6694; V. A. Efimov, A. A. Buryakova, S. V. Reverdattoo, O. G. Chakhmakcheva and Y. A. Ovchinnikov, 1983, Nucleic Acids Res. 11, 8369-8387), to derivatize supports for PNA synthesis (D. A. Stetsenko, E. N. Lubyako, V. K. Potapov, T. L., Azhikina and E. D. Sverdlov, 1996, Tetrahedron Letters 37, 3571-3574), and to prepare supports for the synthesis of oligonucleotides with 3′-alkylphosphate modifications (J. Hovinen, A. Guzaev, A. Azhayev and H. Lonnberg, 1994, Tetrahedron, 7203-7218).

### EXAMPLE 6

### Levulinic Anhydride as a Capping Reagent During the Tandem Synthesis of Multiple Hexanucleotides

In this Example, levulinic anhydride was used as a temporary capping group during the synthesis of three dAAAAAA hexanucleotide sequences in tandem.

Two synthesis columns (˜1 μmole scale) were prepared using 500A long chain alkylamine controlled pore glass (LCAA-CPG) support which had previously been derivatized with 35 μmol/g of 5′-dimethoxytrityl-N-6-benzoyl-2′-deoxyadenosine-3′-O-hydroquinone diacetic acid. The columns were used with a standard 1 μmole scale synthesis cycle and regular synthesis reagents, except a solution of 1 M levulinic anhydride in THF was used as the “Cap A” reagent. The exact weight of support in each column was recorded.

The first column was used as a control and a single dA₆ oligonucleotide was prepared. This was cleaved from the support (NH₄OH, room temperature, 5 minutes) and deprotected (NH₄OH, 16 hours, 55° C.). This yielded 73 A₂₆₀ units of crude product which was 2,100 A₂₆₀ units per gram of support or 58 A₂₆₀ units per μmole of initial nucleoside.

The second column had a dA₆ sequence synthesized on it in the same manner. However, after synthesis the product was left on the support (DMT off). The synthesis column was removed from the synthesizer and treated with 0.5 M hydrazine hydrate in pyridine/acetic acid (3:2 v/v) for 5 minutes. The column was washed with pyridine and then anhydrous acetonitrile. The column was reinstalled on the synthesizer and the Load1234&Cap custom begin procedure was used to attach another dA nucleoside to the 5′-end of the first oligonucleotide. A second dA₆ synthesis and hydrazine treatment was then performed as described above. The product was still left attached to the support and the Load1234&Cap custom begin procedure was run again and a third dA₆ sequence was synthesized (DMT off) to yield a support with three consecutive dA₆ sequences on it. The products were then cleaved from the support (NH₄OH, room temperatue, 5 minutes) and deprotected (NH₄OH, 16 hours, 55° C.). This yielded 163 A₂₆₀ units of crude product which was 4,700 A₂₆₀ units per gram of support or 133 A₂₆₀ units per μmole of initial nucleoside. In comparison to the above control experiment, the amount of product obtained from a single synthesis column was increased ˜1.8 fold.

### EXAMPLE 7

### Levulinic Anhydride as a Capping Reagent During the Tandem Synthesis of Multiple Phosphorothioate 20-mers

In this Example, levulinic anhydride was used as a temporary capping group during the synthesis of two 20 base-long phosphorothioate oligonucleotides.

Two synthesis columns (˜1 μmole scale) were prepared using 500 Å long chain alkylamine controlled pore glass (LCAA-CPG) support which had previously been derivatized with 35 μmol/g of 5′-dimethoxytrityl-N-6-benzoyl-2′-deoxyadenosine-3′-O-hydroquinone diacetic acid. The columns were used with a standard 1 μmole scale phosphorothioate synthesis cycle and regular synthesis reagents, except 0.05 M Beaucage reagent was used as the oxidant and a solution of 1 M levulinic anhydride in THF was used as the “Cap A” reagent. The exact weight of support in each column was recorded.

The first column was used as a control and a single dGCCCAAGCTGGCATCCGTCA oligonucleotide was prepared. This was cleaved from the support (NH₄OH, room temperature, 15 minutes) and deprotected (NH₄OH, 16 hours, 55° C.). This yielded 118 A₂₆₀ units of crude product which was 4,800 A₂₆₀ units per gram of support or 136 A₂₆₀ units per μmole of initial nucleoside.

The second column had a dGCCCAAGCTGGCATCCGTCA sequence synthesized on it in the same manner. However, after synthesis the product was left on the support (DMT off). The synthesis column was removed from the synthesizer and treated with 0.5 M hydrazine hydrate in pyridine/acetic acid (3:2 v/v) for 10 min. The column was washed with pyridine and then anhydrous acetonitrile. The column was reinstalled on the synthesizer and the Load1234&Cap custom begin procedure was used to attach another dA nucleoside to the 5′-end of the first oligonucleotide. A second dGCCCAAGCTGGCATCCGTCA synthesis was then performed. The products were then cleaved from the support (NH₄OH, room temperaure, 15 minutes) and deprotected (NH₄OH, 16 hours, 55° C.). This yielded 188 A₂₆₀ units of crude product which was 8,250 A₂₆₀ units per gram of support or 225 A₂₆₀ units per μmole of initial nucleoside. In comparison to the above control experiment, the amount of product obtained from a single synthesis column was increased ˜1.7 fold.

### EXAMPLE 8

### Synthesis and Separation of Two Different Oligonucleotides by Selective Cleavage of Different Linker Arms

Multiple oligonucleotides can be prepared in tandem on a single solid-phase support. In the previous Examples, these products were released from the support simultaneously to give a mixture of products. This is satisfactory for applications where identical products are required, or when mixtures of different sequences are used together. However, if the different products need to be obtained individually, then a purification using chromatography or electrophoresis is required.

In this Example, a method is described which allows two oligonucleotides to be prepared, in tandem, on a single solid-phase support and then separated from each other without requiring supplementary purification techniques such as any chromatography or electrophoresis. Instead, the oligonucleotides are selectively cleaved from the solid-phase support by exploiting the different cleavage rates of the succinic and hydroquinone-O,O′-diacetic acid linker arms. The two different products are collected as two sequential fractions from the synthesis column during the cleavage step.

This procedure involves the following steps:

A. attachment of the first nucleoside to the support through a relatively resistant linker arm, such as the succinyl linker arm;

B. synthesis of the first oligonucleotide sequence (Oligo #1) by conventional methods;

C. deprotection of the 5′-hydroxyl terminus of Oligo #1;

D. coupling of the next nucleoside onto the end of the first sequence through a more labile linker arm, such as a hydroquinone-O,O′-diacetic acid linker (“Q-Linker”) arm;

E. synthesis of a second oligonucleotide sequence (Oligo #2) by conventional methods;

F. a short (2 min) cleavage step to cleave the labile Q-Linker and release Oligo #2;

G. a second longer (60 min) cleavage step to cleave the more resistant succinic acid linker and release Oligo #1.

As shown in the following experimental section, the amount of contaminating Oligo #1 in the Oligo #2 product is only 2% and the amount of Oligo #2 in Oligo #1 is <0.05%. In both cases, the amount of cross-contamination is less than the N-1 impurities. Therefore, the isolated purity of the two oligonucleotide products is sufficient for them to be used directly in many applications without interference from the other sequence.

The following experimental was used in this Example:

LCAA-CPG (31.5 mg) derivatized with 5′-dimethoxytritylthymidine-3′-O-succinate was placed into a 1 μmol scale synthesis column and installed on a PE/Biosystems 394 DNA synthesizer. The 17 base-long sequence dGTAAAACGACGGCCAGT (Oligo #1) was then prepared using Tr-Off/Man ending option (i.e., product 5′-detritylated and left attached to the support) and conventional 1 μmol scale synthesis cycle and reagents. Trityl analysis indicated an initial nucleoside loading of 39 μmol/g and average coupling yields of 98%.

The synthesizer was then programmed to prepare a second 23 base-long sequence, dCGCCAGGGTTTTCCCAGTCACGA (Oligo #2). The custom Begin Procedure “Load1234&Cap” (see Appendix A) was used to add a 5′-dimethoxytrityl-N⁶-benzoyl-2′-deoxyadenosine-3′-O-hydroquinone-O,O′-diacetic acid nucleoside onto the 5′-hydroxyl terminus of the above sequence. Then the remainder of the sequence was synthesized using the same synthesis cycle, reagents, and Tr-Off/Man ending option as above. Trityl analysis showed an initial nucleoside loading of 28 μmol/g and average coupling yields of 99%.

The synthesis column was removed from the synthesizer and treated with room temperature ammonium hydroxide (1-2 mL) for exactly 2 minutes to cleave Oligo #2 from the support. The NH₄OH was collected in a vial and the column immediately washed with acetonitrile to remove any remaining NH₄OH. The collection vial was sealed, heated overnight at 55° C. to remove protecting groups, placed in a centrifugal evaporator to remove ammonia, and then quantitated by UV. 92 A₂₆₀ units of Oligo #2 was collected. An aliquot (˜0.5 A₂₆₀ units) was desalted by butanol precipitation and analyzed by capillary electrophoresis in triplicate (Table 3). The average full-length product was 80.6%, confirming an average coupling efficiency of 99.0%. The average N-1 impurity was 2.6% and an impurity eluting the same as Oligo #1 (37.4 min) was an average of 2.3%.

The synthesis column was then re-installed on the DNA synthesizer and subjected to an automatic 60 minute cleavage cycle to release Oligo #1 from the support. The collection vial was sealed, heated overnight at 55° C. to remove protecting groups, placed in a centrifugal evaporator to remove ammonia, and then quantitated by UV. 116 A₂₆₀ units of Oligo #1 was collected. An aliquot (˜0.5 A₂₆₀ units) was desalted by butanol precipitation and analyzed by capillary electrophoresis in quadruplicate (Table 3). The average full-length product was 88.1%, confirming an average coupling efficiency of 99.2%. The average N- 1 impurity was 1.4% and cross-contamination with Oligo #2 was undetectable in three out of four analyses. The fourth analysis showed only 0.06% of Oligo #2.

### EXAMPLE 9

### Synthesis of a Duplex (Double-Stranded) DNA Fragment on the Same Support

In this Example, two different oligonucleotide sequences, complementary to each other, were synthesized in tandem on the same support. This Example demonstrates how double-stranded DNA (dsDNA) fragments for gene synthesis or mutagenesis were prepared in a single synthetic step. When the two single-stranded sequences were simultaneously released from the support, they spontaneously hybridized with each other to form the dsDNA fragment shown below.

5′-dTAATACGACTCACTATAGGG-3′ Oligo #1
3′-dATTATGCTGAGTGATATCCC-5′ Oligo #2

Each of the single-stranded sequences was also synthesized separately for comparison using conventional non-tandem synthesis.

Analysis by both by capillary electrophoresis and gel electrophoresis on a native, non-denaturing polyacrylamide gel showed the major product to be the dsDNA fragment. The dsDNA sample was a substrate for T4 polynucleotide kinase and was radioactively labeled with P³². A portion of the radioactively labeled duplex was also shown to be a substrate for T4 DNA ligase and a 40 bp dsDNA fragment was prepared by enzymatic ligation.

The following experiment was used in this Example:

A 1 μmol scale synthesis column containing 5′-dimethoxytrityl-N-2-isobutyryl-2′-deoxyguanosine-3′-O-hydroquinone-O,O′-diacetic acid (27 mg @ 36 μmol/g) was installed on the 394 DNA synthesizer. A 20 base-long sequence, 5′-dTAATACGACTCACTATAGGG-3′ (Oligo #1) was prepared using the Tr-Off/Man ending option. Trityl analysis indicated average coupling yields of 99.5%.

The synthesizer was then programmed to prepare a second 20 base-long sequence, 5′-dCCCTATAGTGAGTCGTATTA-3′ (Oligo #2). The custom Begin Procedure “Load1234&Cap” (see Appendix A) was used to add a 5′-dimethoxytrityl-N⁶-benzoyl-2′-deoxyadenosine-3′-O-hydroquinone-O,O′-diacetic acid nucleoside onto the 5′-hydroxyl terminus of the above sequence and the remainder of the sequence was synthesized using the same synthesis cycle and reagents as above. A Tr-Off/Auto ending option was selected. Trityl analysis showed an initial nucleoside loading of 29 μmol/g and average coupling yields of 99.6%.

The ammonium hydroxide solution collected from the automatic (5 min) ending procedure was heated (16 h, 55° C.) to deprotect the products, evaporated to remove ammonia and quantitated by UV to yield 204 A₂₆₀ units of crude product.

Single-stranded sequences corresponding to Oligos #1 and #2 were also prepared separately by conventional, single syntheses.

An aliquot of the duplex product was desalted by butanol precipitation and analyzed by capillary electrophoresis in quadruplicate. Previously synthesized single-stranded sequences corresponding to Oligo's #1 and 2 were also analyzed by CE for comparison. The two single-stranded 20-mers each migrated as expected for single-stranded oligonucleotides (˜43 min). However, the product prepared from the tandem synthesis migrated much slower, at ˜67 min, indicating a duplex and not single-stranded structure. The average amount of full-length duplex product was 84.7% and no impurities greater than 1% were present with migration times corresponding to the single-stranded 20-mer sequences.

Samples of the tandem dsDNA synthesis and the separate single-stranded sequences were 5′-phosphorylated and radioactively labeled with T4 DNA kinase. The duplex product was also treated with DNA ligase to self-ligate the 20 bp dsDNA product into a 40 bp dsDNA product.

These samples were run on polyacrylamide gels under both native, non-denaturing conditions and denaturing conditions. The non-denaturing gel showed the duplex product had the expected slower mobility than the single-stranded product. The denaturing gel showed that the tandem synthesis products had the expected identical mobility as the single-stranded sequences as well as the presence of a 40 bp self-ligation product.

### EXAMPLE 10

### Tandem Synthesis of Fluorescently Labeled PCR Primers

DNA fragments labeled with non-radioactive fluorescent dye labels are widely used for the automated detection and analysis of DNA fragments. Introduction of the fluorescent label is commonly performed by PCR amplification of the target DNA.

In this method, one of the two synthetic oligonucleotide PCR primers contains a 5′-terminal fluorescent dye. Since a pair of different priming sequences is conventionally required for PCR amplification, synthesis of two PCR primers in tandem is a useful application.

An ABI 394 DNA synthesizer was configured for tandem synthesis. HEX, FAM, and TET fluorescent dye phosphoramidite reagents from Applied Biosystems were installed on spare base positions. Three pairs of PCR primers for amplification of known microsatellite markers from mouse genomic DNA were prepared (one pair of primers per tandem synthesis):

D14Mit110 microsatellite marker
Primer #1 (22-mer): dCATGAATAAGAACGAAAAGGGC
Primer #2 (26-mer): HEX-dGTAGGAGAGAACAACTGTCTTCTGC
D1Mit46 microsatellite marker
Primer #1 (20-mer): dCACGGGTGCTCTATTTGGAA
Primer #2 (23-mer): FAM-dAGTCAGTCAGGGCTACATGATG
D4Mit59 microsatellite marker
Primer #1 (23-mer): dTATCCAACACATTTATGTCTGCG
Primer #2 (22-mer): TET-dAGAGTTTGGTCTCTTCCCCTG

After tandem synthesis, the products were cleaved from the support and deprotected in ammonium hydroxide (55°, 16 h). The three crude mixtures were then analyzed by capillary gel electrophoresis to determine the following ratios of Primer #1 to Primer #2: D14Mit110, 61.2: 27.2 (11.7% of unlabeled Primer #2 was also present as a failure sequence); D1Mit46, 53.5: 46.5; D4Mit59, 64.8: 35.2.

The crude mixtures were then used to amplify mouse genomic DNA and the fluorescent products were analyzed on an ABI 377 DNA sequencer using GeneScan software. For comparison, each of the six primers was also purified by preparative gel electrophoresis and then each primer pair was reconstituted using a 1:1 ratio of purified primers. The purified primer sets were then used to amplify the same mouse genomic DNA. No significant difference was observed between the crude tandem mixtures and the individually purified primers.

### EXAMPLE 11

### Tandem Synthesis of a Mixture of Trinucleotide-5′-phosphates

The synthesis of mixtures of short oligonucleotides suitable for enzymatic assembly into longer sequences is useful for preparing DNA libraries. However, enzymatic ligation conventionally requires the presence of a 5′-phosphate on the oligonucleotides. Such oligonucleotide mixtures can be made by including commercially available phosphoramidite reagents (“Phosphate On”) in combination with tandem synthesis of the present invention.

An ABI 394 DNA synthesizer was configured for tandem synthesis and a solution of “Phosphate On” reagent was installed on spare base position #5. The sequence 5TTT was then prepared using the Trityl-Off/Manual end procedure, i.e. no cleavage from the support. The Load1234&Cap begin procedure was used to add a T nucleoside to the 5′-end of the existing sequence and the sequence 5CCT was prepared. This process was then repeated twice more to prepare the sequences 5GGT and 5AAT. Trityl analysis indicated an average coupling efficiency of 98.8% for the entire synthesis. Then the four tandem synthesized trinucleotide-5′-phosphates were cleaved from the synthesis column (NH₄OH, 60 min) and deprotected (NH₄OH, 16 h, 55°) to yield 49 A₂₆₀ units of a mixture containing d(pAAT), d(pGGT), d(pCCT) and d(pTTT).

All publications, patents and patent applications referred to herein are incorporated by reference in their entirety to the same extent as if each individual publication, patent or patent application was specifically and individually indicated to be incorporated by reference in its entirety.

TABLE 1
Results From Two Phosphorothioate Oligonucleotide 20-mer
Syntheses Linked in Tandem on a Reusable Solid-Phase Support

| | | | Crude | |
|---|---|---|---|---|
| | | | Product | Increase |
| | | Amount of | (A₂₆₀ | (relative |
| | Initial | crude product | units/ | to control |
| Experiment | nucleoside | (A₂₆₀ units) | μmole) | expt.) |
| Control expt., | 1.1 | 143 | 131 | — |
| single synthesis | | | | |
| Recycling expt., | 1 | 215 | 213 | 1.63 |
| 1^{st} use | | | | |
| Recycling expt., | 0.87 | 218 | 251 | 1.92 |
| 2^{nd} use | | | | |
| Recycling expt., | 0.75 | 134 | 178 | 1.36 |
| 3^{rd} use | | | | |
| Recycling expt., | 0.85 | 152 | 179 | 1.37 |
| 4^{th} use | | | | |
| Recycling expt., | 0.76 | 134 | 176 | 1.35 |
| 5^{th} use | | | | |

TABLE 2
Results From Multiple Tandem Syntheses of an M13 24 Base-Long
Sequencing Primer on 1,000 Å CPG

| | | Amount of | Crude | | % Full |
|---|---|---|---|---|---|
| | | crude | Product | Increase | length |
| # of | Total | product | A₂₆₀ | (relative to | product |
| Syntheses | Bases | (A₂₆₀ units) | units/μmole | control expt.) | (CE) |
| 1 | 24 | 37.7 | 168 | — | 61.0% |
| 2 | 48 | 64.3 | 245 | 1.5 | 68.5% |
| 3 | 72 | 78.8 | 356 | 2.1 | 69.3% |
| 4 | 96 | 105 | 425 | 2.5 | 65.5% |

TABLE 3
Capillary Electrophoresis Data of Oligonucleotide Separated by
Selective Cleavage

| | Full Length Product | | | N-1 Impurity | | Cross-Contamination | |
|---|---|---|---|---|---|---|---|
| | M.T.^{a} (min) | Area %^{b} | A.Y. (%)^{c} | M.T.^{a} (min) | Area %^{b} | M.T.^{a} (min) | Area %^{b} |
| Oligo #1 | 37.788 | 89.8160 | 99.33 | 35.981 | 1.7668 | 43.375 | 0.0561 |
| | 38.138 | 90.6246 | 99.39 | 35.920 | 1.2095 | N.D.^{d} | N.D.^{d} |
| | 40.345 | 86.0400 | 99.06 | 39.127 | 1.5909 | N.D.^{d} | N.D.^{d} |
| | 39.161 | 85.7639 | 99.04 | 37.501 | 1.0979 | N.D.^{d} | N.D.^{d} |
| Averages | | 88.0611 | 99.21 | | 1.4163 | | 0.0140 |
| Oligo #2 | 43.363 | 81.4148 | 99.07 | 42.635 | 2.8839 | 37.376 | 2.2904 |
| | 44.722 | 80.9965 | 99.05 | 44.302 | 1.0925 | 38.566 | 2.1948 |
| | 46.041 | 79.4895 | 98.96 | 45.339 | 3.8007 | 39.723 | 2.3105 |
| Averages | | 80.6336 | 99.03 | | 2.5924 | | 2.2652 |

^{a}Migration Time
^{b}Corrected for mobility
^{c}Average coupling yield based on Area %
^{d}Not detected

[image]

What is claimed is:

1. A process for producing at least two molecules of interest on a solid support material, the process comprising the steps of:
(i) reacting a support having Formula I:
[SUPPORT]-Z¹-Y¹ (I)
wherein:
Z¹ is N or O;
and Y¹ is —H when Z¹ is O or Y¹ is —(R)(H) when Z¹ is N wherein R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group;
with a compound having Formula II:
H-A¹-B¹ (II)
wherein:
A¹ is a linker moiety having the formula 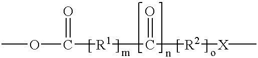
wherein:
R¹ and R² are the same or different and each is an organic moiety;
m is 0 or 1;
n is 0 or 1;
o is 0 or 1; and
X is O or N(R) wherein R is as defined above; and
B¹ is hydrogen or a protecting group;
to produce a material having Formula III:
[SUPPORT]-Z¹-A¹-B¹ (III)
(ii) conducting at least one synthetic reaction on the material having Formula III to produce a material having Formula IV: 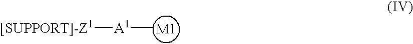
wherein M1 is a precursor to one of the at least two molecules of interest;
(iii) reacting the material of Formula IV with a compound having Formula V:
H-A²-B² (V)
wherein:
A² is a moiety selected from the group defined above for A¹; and
B² is a moiety selected from the group defined above for B¹;
to produce a material having Formula VI: 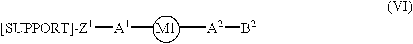
(iv) conducting at least one synthetic reaction on the material having Formula VI to produce a material having Formula VII: 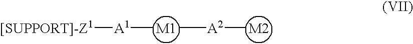
wherein M2 is a precursor of the other of the at least two molecules of interest; and
(v) subjecting the material of Formula VII to a cleavage step to release at least one of the at least two molecules of interest from the support material.

2. A process for producing at least two oligonucleotides on a solid support material, each oligonucleotide having a predetermined sequence, the process comprising the steps of:
(i) reacting a support having Formula I:
[SUPPORT]-Z¹-Y¹ (I)
wherein:
Z¹ is N or O;
and Y¹ is —H when Z¹ is O or Y¹ is —(R)(H) when Z¹ is N wherein R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group;
with a compound having Formula II:
H-A¹-B¹ (II)
wherein:
A¹ is a moiety having the formula 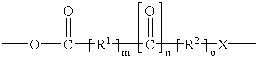
wherein:
R¹ is an organic moiety;
R² is a nucleoside moiety or a nucleotide moiety;
m is 0 or 1;
n is 0 or 1;
o is 0 or 1; and
X is O or N(R) wherein R is as defined above; and
B¹ is hydrogen or a protecting group;
to produce a material having Formula III:
[SUPPORT]-Z¹-A¹-B¹ (III)
(ii) conducting at least one synthetic reaction on the material having Formula III to produce a material having Formula IV: 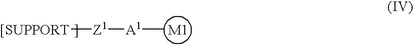
wherein M1 comprises at least a portion of the predetermined sequence of one of the at least two oligonucleotides of interest;
(iii) reacting the material of Formula IV with a compound having Formula V:
H-A²-B² (V)
wherein:
A² is a moiety selected from the group defined above for A¹; and
B² is a moiety selected from the group defined above for B¹;
to produce a material having Formula VI: 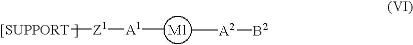
(iv) conducting at least one synthetic reaction on the material having Formula VI to produce a material having Formula VII: 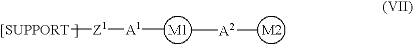
wherein M2 comprises at least a portion of the predetermined sequence of the other of the at least two oligonucleotides of interest; and
(v) subjecting the material of Formula VII to a cleavage step to release at least one of the at least two oligonucleotides of interest from the support material.

3. The process defined in claim 2, wherein Step (v) comprises cleaving the at least two oligonucleotides of interest from the support material.

4. The process defined in claim 3, wherein, after Step (v), the at least two oligonucleotides of interest are joined to each other.

5. The process defined in claim 3, wherein, after Step (v), the at least two oligonucleotides of interest are independent with respect to each other.

6. The process defined in claim 2, wherein R² is a moiety having one of the following formulae: 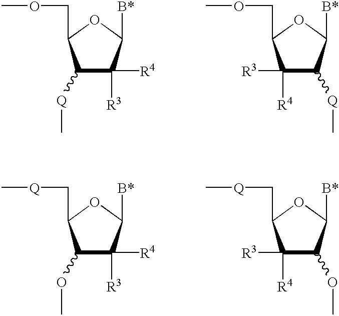
wherein:
R³ and R⁴ are the same or different and each is selected from the group consisting of hydrogen, a halide, hydroxyl, NH₂, NHR, NR₂ and OR* wherein R* is a protecting group or is R as defined above;
B* is a nucleic acid base; and
Q is selected from the group consisting of O, a substituted or unsubstituted phosphate group, a substituted or unsubstituted phosphonate group, a substituted or unsubstituted phosphite group, a substituted or unsubstituted phosphorothioate group, a substituted or unsubstituted phosphorodithioate group, a substituted or unsubstituted C₁-C₂₀ alkyl group, a substituted or unsubstituted C₅-C₃₀ aryl group, a substituted or unsubstituted C₅-C₄₀ alkylaryl group, NH and NR wherein R is as defined above.

7. The process defined in claim 6, wherein Q is O and R³ and R⁴ are the same or different and each is selected from the group consisting of hydrogen and OR* wherein R* is H or a protecting group.

8. The process defined in claim 2, wherein R¹ is a moiety having the formula: 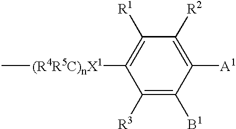
wherein: R¹, R² and R³ are the same or different and are selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; R⁴ and R⁵ are the same or different and are selected from the group consisting of hydrogen, halogen, a substituted or unsubstituted C₁-C₂₀ alkyl group, a substituted or unsubstituted C₅-C₃₀ aryl group and a substituted or unsubstituted C₅-C₄₀ alkylaryl group; X¹ is selected from the group consisting of —O—, —S—, —C(O)—, —S(O)₂— and —N(R)—; R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group; n is 0, 1 or 2; and one of A¹ and B¹ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of A¹ and B¹ has the formula: 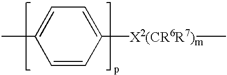
wherein p is 0 or 1, X² is selected from the group consisting of —O—, —S—, —C(O)—, —S(O)₂— and —N(R)—, R is selected hydrogen, a substituted or unsubstituted C₁-C₂₀ alkyl group, a substituted or unsubstituted C₅-C₃₀ aryl group and a substituted or unsubstituted C₅-C₄₀ alkylaryl group, R⁶ and R⁷ are the same or different and are selected from the group consisting of a hydrogen, halogen, substituted or unsubstituted C₁-C₂₀ alkyl group, a substituted or unsubstituted C₅-C₃₀ aryl group and a substituted or unsubstituted C₅-C₄₀ alkylaryl group, and m is 0, 1 or 2.

9. The process defined in claim 2, R¹ is a moiety having the formula:
—(R⁴R⁵C)ₙ—(Y)ₒ—(CR⁶R⁷)ₘ—
wherein R⁴, R⁵, R⁶ and R⁷ are the same or different and are selected from the group consisting of hydrogen, halogen, a substituted or unsubstituted C₁-C₂₀ alkyl group, a substituted or unsubstituted C₅-C₃₀ aryl group and a substituted or unsubstituted C₅-C₄₀ alkylaryl group, Y is selected from the group consisting of O, S, SO₂ and O—((CH₂)ₗ—O)_{q}, l is an integer less than or equal to 60, q is an integer in the range of 1-1000, n and m are the same or different and are 0, 1 or 2, and o is 0 or 1.

10. The process defined in claim 2, wherein R¹ is a moiety selected from the group consisting of the following formulae:

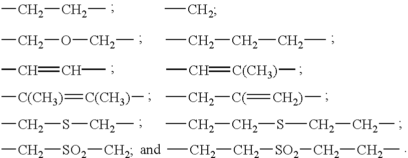

11. The process defined in claim 2, wherein the compounds of Formula II and Formula V are the same.

12. The process defined in claim 2, wherein the compounds of Formula II and Formula V are different.

13. The process defined in claim 2, wherein, prior to Step (v), Steps (iii) and (iv) are repeated at least once.

14. The process defined in claim 2, wherein Step (v) comprises cleaving all oligonucleosides produced on the support material.

15. A material having Formula VI: 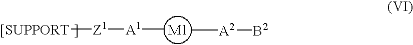
wherein:
Z¹ is O or N(R) wherein R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group;
A¹ and A² are the same or different and each is a linker moiety having the formula 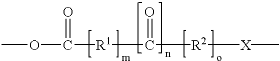
wherein:
R¹ and R² are the same or different and each is an organic moiety;
m is 0 or 1;
n is 0 or 1;
o is 0 or 1; and
X is O or NR wherein R is as defined above;
B² is hydrogen or a protecting group; and
M1 is a precursor to a first molecule of interest.

16. The material defined in claim 15, wherein R² is a moiety having one of the following formulae: 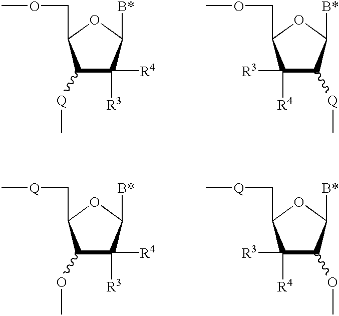
wherein:
R³ and R⁴ are the same or different and each is selected from the group consisting of hydrogen, a halide, hydroxyl, NH₂, NHR, NR₂ and OR* wherein R* is a protecting group or is R as defined above;
B* is a nucleic acid base; and
Q is selected from the group consisting of O, a substituted or unsubstituted phosphate group, a substituted or unsubstituted phosphonate group, a substituted or unsubstituted phosphite group, a substituted or unsubstituted phosphorothioate group, a substituted or unsubstituted phosphorodithioate group, a substituted or unsubstituted C₁-C₂₀ alkyl group, a substituted or unsubstituted C₅-C₃₀ aryl group, a substituted or unsubstituted C₅-C₄₀ alkylaryl group, NH and NR wherein R is as defined above.

17. The material defined in claim 16, wherein Q is O and R³ and R⁴ are the same or different and each is selected from the group consisting of hydrogen and OR* wherein R* is H or a protecting group.

18. The material defined in claim 15, wherein R¹ is a moiety having the formula: 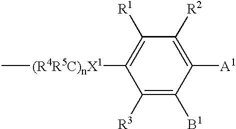
wherein: R¹, R² and R³ are the same or different and are selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; R⁴ and R⁵ are the same or different and are selected from the group consisting of hydrogen, halogen, a substituted or unsubstituted C₁-C₂₀ alkyl group, a substituted or unsubstituted C₅-C₃₀ aryl group and a substituted or unsubstituted C₅-C₄₀ alkylaryl group; X¹ is selected from the group consisting of —O—, —S—, —C(O)—, —S(O)₂— and —N(R)—; R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group; n is 0, 1 or 2; and one of A¹ and B¹ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of A¹ and B¹ has the formula: 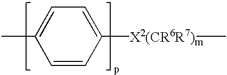
wherein p is 0 or 1, X² is selected from the group consisting of —O—, —S—, —C(O)—, —S(O)₂— and —N(R)—, R is selected hydrogen, a substituted or unsubstituted C₁-C₂₀ alkyl group, a substituted or unsubstituted C₅-C₃₀ aryl group and a substituted or unsubstituted C₅-C₄₀ alkylaryl group, R⁶ and R⁷ are the same or different and are selected from the group consisting of hydrogen, halogen, a substituted or unsubstituted C₁-C₂₀ alkyl group, a substituted or unsubstituted C₅-C₃₀ aryl group and a substituted or unsubstituted C₅-C₄₀ alkylaryl group, and m is 0, 1 or 2.

19. The material defined in claim 15, R¹ is a moiety having the formula:
—(R⁴R⁵C)ₙ—(Y)ₒ—(CR⁶R⁷)ₘ—
wherein R⁴, R⁵, R⁶ and R⁷ are the same or different and are selected from the group consisting of hydrogen, halogen, a substituted or unsubstituted C₁-C₂₀ alkyl group, a substituted or unsubstituted C₅-C₃₀ aryl group and a substituted or unsubstituted C₅-C₄₀ alkylaryl group, Y is selected from the group consisting of O, S, SO₂ and O—((CH₂)ₗ—O)_{q}, l is an integer less than or equal to 60, q is an integer in the range of 1-1000, n and m are the same or different and are 0, 1 or 2, and o is 0 or 1.

20. The material defined in claim 15, wherein R is a moiety selected from the group consisting of the following formulae:

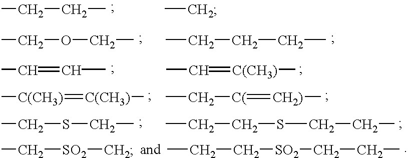

21. A material having Formula VII: 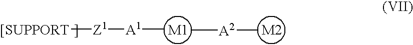
wherein:
Z¹ is O or N(R) wherein R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group;
A¹ and A² are the same or different and each is a linker moiety having the formula 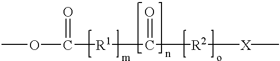
wherein:
R¹ and R² are the same or different and each is an organic moiety;
m is 0 or 1;
n is 0 or 1;
o is 0 or 1; and
X is O or NR wherein R is as defined above;
B² is hydrogen or a protecting group; and
M1 is a precursor to a first molecule of interest; and
M2 is a precursor to a second molecule of interest.

22. The material defined in claim 21, wherein R² is a moiety having one of the following formulae: 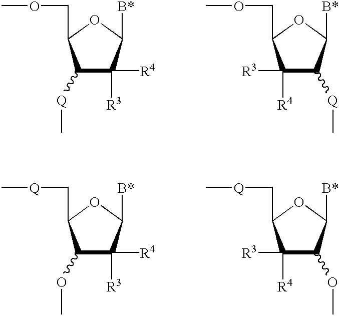
wherein:
R³ and R⁴ are the same or different and each is selected from the group consisting of hydrogen, a halide, hydroxyl, NH₂, NHR, NR₂ and OR* wherein R* is a protecting group or is R as defined above;
B* is a nucleic acid base; and
Q is selected from the group consisting of O, a substituted or unsubstituted phosphate group, a substituted or unsubstituted phosphonate group, a substituted or unsubstituted phosphite group, a substituted or unsubstituted phosphorothioate group, a substituted or unsubstituted phosphorodithioate group, a substituted or unsubstituted C₁-C₂₀ alkyl group, a substituted or unsubstituted C₅-C₃₀ aryl group, a substituted or unsubstituted C₅-C₄₀ alkylaryl group, NH and NR wherein R is as defined above.

23. The material defined in claim 22, wherein Q is O and R³ and R⁴ are the same or different and each is selected from the group consisting of hydrogen and OR* wherein R* is H or a protecting group.

24. The material defined in claim 21, wherein R¹ is a moiety having the formula: 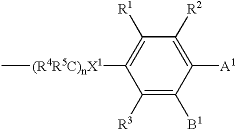
wherein: R¹, R² and R³ are the same or different and are selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; R⁴ and R⁵ are the same or different and are selected from the group consisting of hydrogen, halogen, a substituted or unsubstituted C₁-C₂₀ alkyl group, a substituted or unsubstituted C₅-C₃₀ aryl group and a substituted or unsubstituted C₅-C₄₀ alkylaryl group; X¹ is selected from the group consisting of —O—, —S—, —C(O)—, —S(O)₂— and —N(R)—; R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group; n is 0, 1 or 2; and one of A¹ and B¹ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of A¹ and B¹ has the formula: 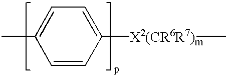
wherein p is 0 or 1, X² is selected from the group consisting of —O—, —S—, —C(O)—, —S(O)₂— and —N(R)—, R is selected hydrogen, a substituted or unsubstituted C₁-C₂₀ alkyl group, a substituted or unsubstituted C₅-C₃₀ aryl group and a substituted or unsubstituted C₅-C₄₀ alkylaryl group, R⁶ and R⁷ are the same or different and are selected from the group consisting of a hydrogen, halogen, substituted or unsubstituted C₁-C₂₀ alkyl group, a substituted or unsubstituted C₅-C₃₀ aryl group and a substituted or unsubstituted C₅-C₄₀ alkylaryl group, and m is 0, 1 or 2.

25. The material defined in claim 21, wherein R¹ is a moiety having the formula:
—(R⁴R⁵C)ₙ—(Y)ₒ—(CR⁶R⁷)ₘ—
wherein R⁴, R⁵, R⁶ and R⁷ are the same or different and are selected from the group consisting of hydrogen, halogen, a substituted or unsubstituted C₁-C₂₀ alkyl group, a substituted or unsubstituted C₅-C₃₀ aryl group and a substituted or unsubstituted C₅-C₄₀ alkylaryl group, Y is selected from the group consisting of O, S, SO₂ and O—((CH₂)ₗ—O)_{q}, l is an integer less than or equal to 60, q is an integer in the range of 1-1000, n and m are the same or different and are 0, 1 or 2, and o is 0 or 1.

26. The material defined in claim 21, wherein R¹ is a moiety selected from the group consisting of the following formulae:

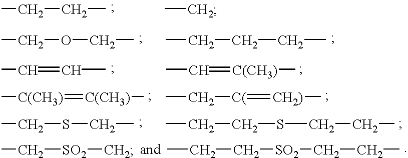

27. A process for producing at least two oligonucleotides on a solid support material, each oligonucleotide having a predetermined sequence, the process comprising the steps of:
(i) conducting at least one synthetic reaction on a material having Formula III
[SUPPORT]-Z¹-A¹-B¹ (III)
wherein:
Z¹ is O or N(R) wherein R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group;
A¹ is a moiety having the formula 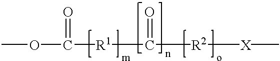
R¹ is an organic moiety;
R² is a nucleoside moiety or a nucleotide moiety;
m is 0 or 1;
n is 0 or 1;
o is 0 or 1; and
X is O or N(R) wherein R is as defined above; and
B¹ is hydrogen or a protecting group;
to produce a material having Formula IV: 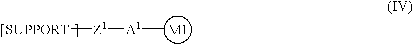
wherein M1 comprises at least a portion of the predetermined sequence of one of the at least two oligonucleotides of interest;
(ii) reacting the material of Formula IV with a compound having Formula V:
H-A²-B² (V)
wherein:
A² is a moiety selected from the group defined above for A¹; and
B² is a moiety selected from the group defined above for B¹;
to produce a material having Formula VI: 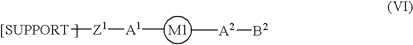
(iii) conducting at least one synthetic reaction on the material having Formula VI to produce a material having Formula VII: 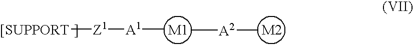
wherein M2 comprises at least a portion of the predetermined sequence of the other of the at least two oligonucleotides of interest; and
(iv) subjecting the material of Formula VII to a cleavage step to release at least one of the at least two oligonucleotides of interest from the support material.

28. The process defined in claim 27, wherein Step (iv) comprises cleaving the at least two oligonucleotides of interest from the support material.

29. The process defined in claim 27, wherein, after Step (iv), the at least two oligonucleotides of interest are joined to each other.

30. The process defined in claim 27, wherein, after Step (v), the at least two oligonucleotides of interest are independent with respect to each other.

31. The process defined in claim 27, wherein R² is a moiety having one of the following formulae: 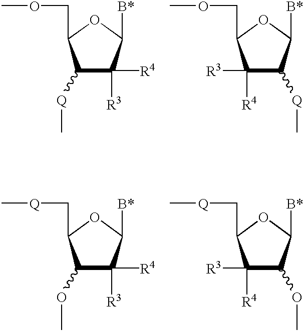
wherein:
R³ and R⁴ are the same or different and each is selected from the group consisting of hydrogen, a halide, hydroxyl, NH₂, NHR, NR₂ and OR* wherein R* is a protecting group or is R as defined above;
B* is a nucleic acid base; and
Q is selected from the group consisting of O, a substituted or unsubstituted phosphate group, a substituted or unsubstituted phosphonate group, a substituted or unsubstituted phosphite group, a substituted or unsubstituted phosphorothioate group, a substituted or unsubstituted phosphorodithioate group, a substituted or unsubstituted C₁-C₂₀ alkyl group, a substituted or unsubstituted C₅-C₃₀ aryl group, a substituted or unsubstituted C₅-C₄₀ alkylaryl group, NH and NR wherein R is as defined above.

32. The process defined in claim 31, wherein Q is O and R³ and R⁴ are the same or different and each is selected from the group consisting of hydrogen and OR* wherein R* is H or a protecting group.

33. The process defined in claim 27, wherein R¹ is a moiety having the formula: 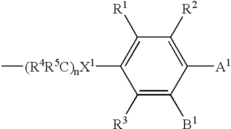
wherein: R¹, R² and R³ are the same or different and are selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; R⁴ and R⁵ are the same or different and are selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group; X¹ is selected from the group consisting of —O—, —S—, —C(O)—, —S(O)₂— and —N(R)—; R is selected from the group consisting of hydrogen, a substituted or unsubstituted C₁-C₂₀ alkyl group, a substituted or unsubstituted C₅-C₃₀ aryl group and a substituted or unsubstituted C₅-C₄₀ alkylaryl group; n is 0, 1 or 2; and one of A¹ and B¹ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of A¹ and B¹ has the formula: 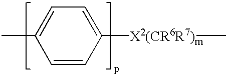
wherein p is 0 or 1, X² is selected from the group consisting of —O—, —S—, —C(O)—, —S(O)₂— and —N(R)—, R is selected hydrogen, a substituted or unsubstituted C₁-C₂₀ alkyl group, a substituted or unsubstituted C₅-C₃₀ aryl group and a substituted or unsubstituted C₅-C₄₀ alkylaryl group, R⁶ and R⁷ are the same or different and are selected from the group consisting of a substituted or unsubstituted C₁-C₂₀ alkyl group, a substituted or unsubstituted C₅-C₃₀ aryl group and a substituted or unsubstituted C₅-C₄₀ alkylaryl group, and m is 0, 1 or 2.

34. The process defined in claim 27, wherein R¹ is a moiety having the formula:
—(R⁴R⁵C)ₙ—(Y)ₒ—(CR⁶R⁷)ₘ—
wherein R⁴, R⁵, R⁶ and R⁷ are the same or different and are selected from the group consisting of hydrogen, halogen, a substituted or unsubstituted C₁-C₂₀ alkyl group, a substituted or unsubstituted C₅-C₃₀ aryl group and a substituted or unsubstituted C₅-C₄₀ alkylaryl group, Y is selected from the group consisting of O, S, SO₂ and O—((CH₂)ₗ—O)_{q}, l is an integer less than or equal to 60, q is an integer in the range of 1-1000, n and m are the same or different and are 0, 1 or 2, and o is 0 or 1.

35. The process defined in claim 27, wherein R¹ is a moiety selected from the group consisting of the following formulae:

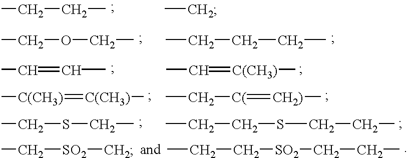

36. The process defined in claim 27, wherein A¹ and A² are the same.

37. The process defined in claim 26, wherein A¹ and A² are different.

38. The process defined in claim 26, wherein, prior to Step (iv), Steps (ii) and (iii) are repeated at least once.

39. The process defined in claim 26, wherein Step (v) comprises cleaving all oligonucleotides produced on the support material.
